(19) United States
(12) Patent Application Publication
(10) Pub. No.: US 2026/0137863 A1
(43) Pub. Date: May 21, 2026

(54) WIRELESS FLUID SENSOR FOR INLINE LIQUID SENSING IN MEDICAL SYSTEMS

(71) Applicant: Analog Devices, Inc., Wilmington, MA (US)

(72) Inventors: David Frank Bolognia, Charlestown, MA (US); Jackson Coole, Missouri City, TX (US); Liam Riordan, Limerick (IE); Jeremy Georges, Colorado Springs, CO (US); Aleksander Franz, Winchester, MA (US); Lei Gu, Lexington, MA (US)

(21) Appl. No.: 19/393,495

(22) Filed: Nov. 18, 2025

Related U.S. Application Data
(60) Provisional application No. 63/722,521, filed on Nov. 19, 2024.

(51) Int. Cl. A61M 5/168; A61M 5/142

(52) U.S. Cl. CPC A61M 5/16831; A61M 5/142; A61M 2005/14208; A61M 2005/16863; A61M 2205/3334; A61M 2205/3368; A61M 2205/3592; A61M 2205/50; A61M 2205/8243

(57) ABSTRACT

A fluid sensor assembly including a fluid sensor, an antenna, and a processor in wireless communication with the fluid sensor assembly and comprising a reader. The fluid sensor can be positioned along a fluid flow line. The fluid sensor can be configured to output one or more output signals indicative of one or more properties of the fluid flowing through the fluid sensor. The antenna of the fluid sensor assembly is configured to transmit the output signals to the reader of the processor; and the processor is configured to process the signals received by the reader.

### CROSS-REFERENCE TO RELATED APPLICATIONS

This application claims priority to U.S. App. No. 63/722,521, which is titled “FLOW SENSOR ON GLASS AND INSERT MOLDED INTO FITTINGS” and was filed on Nov. 19, 2024, which is incorporated herein by reference in its entirety.

### TECHNICAL FIELD

The application is generally directed to flow sensors and flow sensor glass sub-assemblies.

### BACKGROUND

Flow sensors or meters can measure how much fluid (e.g., liquid, gas, etc.) is flowing through a tube and/or other conduit. In medical devices, flow sensors can be used to measure and/or control the flow of gases (e.g., oxygen) and/or fluids (e.g., medication). For example, flow sensors are crucial for monitoring and controlling fluid flow in various medical devices and procedures, including dialysis, mechanical ventilation, intravenous fluid delivery, application of anesthesia, etc. Flow sensors should provide accurate and precise measurements to ensure patients receive correct dosages. Currently available flow sensors are bulky, expensive, and/or not customizable, and are therefore not a viable option for many medical applications and/or procedures.

Moreover, infusion pumps are critical in the administration of medications, nutrients, and other fluids to patients in a controlled manner. However, a lack of flow continuity (i.e., inconsistent rate of delivery) can result in serious clinical consequences, including delay of therapy, over-infusion, and/or under-infusion. While current infusion pumps can reach a relatively good rate of accuracy from motion control algorithms and sensor inputs, flow rate accuracy can be affected by fluid characteristics (viscosity, temperature, density specific weight, volume, pressure, etc.) and/or intravenous set characteristics (inner diameter, material) that can be difficult to address in real-time. Therefore, there is a need for an efficient, reliable, and user-friendly solution to accurately quantify fluid delivery.

### SUMMARY

The innovations described in the claims each have several aspects, no single one of which is solely responsible for its desirable attributes. Without limiting the scope of the claims, some prominent features of this disclosure will now be briefly described.

There is provided in accordance with one aspect of the present disclosure, a system for measuring one or more properties of a fluid. The system can include a fluid sensor assembly including a fluid sensor and an antenna, wherein the fluid sensor is configured to be removably positioned along a fluid flow line; a processor in wireless communication with the fluid sensor assembly and including a reader; wherein the fluid sensor is configured to output one or more output signals indicative of one or more properties of the fluid flowing through the fluid sensor; wherein the antenna of the fluid sensor assembly is configured to transmit the output signals to the reader of the processor, and wherein the processor is configured to process the signals received by the reader.

In some aspects, the fluid sensor can include a flow path in communication with the fluid flow line, and one or more resistor pairs at least partially exposed to the flow path.

The fluid sensor assembly can be configured to drive the one or more resistor pairs and measure a change in resistance as the fluid flows through the flow path.

In some cases, the one or more properties of the fluid can include at least one of flow rate, flow direction, and temperature.

In some aspects, the one or more output signals can be indicative of a presence of air bubbles or occlusions in the system.

The reader can be configured to generate an electromagnetic field configured to induce a current in a coil of the antenna, and wherein the current is configured to power the fluid sensor.

In some cases, the reader and the antenna can be positioned about 25 cm or less from each other.

In some aspects, the fluid sensor assembly can include an analog-to-digital converter (ADC) configured to convert the output signals to digital signals.

The system can include an infusion pump.

In some cases, the processor can be positioned inside the infusion pump and is further configured to control operational settings of the infusion pump based at least in part on the processed signals.

In some aspects, the fluid flow line can include a first fluid line configured to place a fluid container in communication with the fluid sensor assembly; a second fluid line configured to place the fluid sensor assembly in communication with a patient.

The fluid sensor can include at least two substrates stacked on each other; a flow channel formed between the at least two substrates; a through substrate vias extending through at least a portion of at least one of the two substrates; one or more sensing elements along the flow channel; and a chip mounted to at least one of the two substrates; wherein the chip and the one or more sensing elements are in electrical communication via the through substrate vias.

There is also provided in accordance with one aspect of the present disclosure, a system for measuring one or more properties of a fluid. The system can include an infusion pump; a fluid container configured to contain a fluid; a fluid sensor assembly including, a fluid sensor including a flow path and one or more sensing elements at least partially exposed to the flow path, wherein the fluid from the fluid container is configured to flow through the flow path, and a first processor in communication with the fluid sensor and including a signal conditioning circuitry, and an antenna; a first fluid line configured to place the fluid container in communication with the flow path of the fluid sensor; a second fluid line configured to place flow path of the fluid sensor in communication with a patient; and a second processor in communication with the fluid sensor assembly and including a reader, wherein the second processor is positioned on the infusion pump; wherein the fluid sensor is configured to output one or more output signals representative of a change in resistance of the one or more sensing elements, and wherein the change of resistance of the one or more sensing element is indicative of one or more properties of the fluid flowing through the flow path of the fluid sensor; wherein the signal conditioning circuitry is configured to receive and process the one or more output signals from the fluid sensor, wherein the antenna of the first processor is configured to transmit the signals processed by the signal conditioning circuitry across the infusion pump; wherein the reader of the second processor is configured to receive the signals transmitted by the antenna of the first processor; and wherein the second processor is configured to process the signals received by the reader.

In some aspects, the one or more sensing elements can include one or more thermistor pairs.

The signal conditioning circuitry can include an analog front end (AFE).

In some cases, the antenna of the first processor can include a near-field communication (NFC) antenna.

In some aspects, the reader of the second processor can include a near-field communication (NFC) reader.

The first processor can includes a data buffer.

In some cases, the one or more properties of the fluid can include at least one of flow rate, flow direction, and temperature.

In some aspects, the one or more output signals can be further indicative of a presence of air bubbles or occlusions in the system.

The reader can be configured to generate an electromagnetic field configured to induce a current in a coil of the antenna, and wherein the current is configured to power at least one of the signal conditioning circuitry or the fluid sensor.

In some cases, the reader and the antenna can be positioned about 25 cm or less from each other.

In some aspects, the fluid sensor can be configured to be removably positioned along a fluid flow path of an IV line extending from a fluid container to a patient.

The fluid sensor assembly can include an analog-to-digital converter (ADC) configured to convert the signals processed by the signal conditioning circuitry to digital signals.

In some cases, the signal conditioning circuitry can include an analog front end (AFE).

In some aspects, the fluid sensor can include at least two substrates stacked on each other, wherein the flow path is formed between the at least two substrates; a through substrate vias extending through at least a portion of at least one of the two substrates; and a chip mounted to at least one of the two substrates; wherein the chip and the one or more sensing elements are in electrical communication via the through substrate vias.

There is also provided in accordance with one aspect of the present disclosure, a system for measuring one or more properties of a fluid. The system can include an infusion pump; a fluid sensor assembly including a fluid sensor, a fluid sensor signal conditioning circuitry, and an antenna; a processor in wireless communication with the fluid sensor assembly and including a reader, wherein the processor is positioned inside the infusion pump; wherein the fluid sensor is configured to output one or more output signals indicative of one or more properties of a fluid flowing through the fluid sensor; wherein the signal conditioning circuitry is configured to receive and condition the one or more output signals from the fluid sensor, wherein the antenna of the fluid sensor assembly is configured to transmit the signals processed by the signal conditioning circuitry across a wall of the infusion pump; wherein the reader of the processor is configured to receive the signals transmitted by the antenna of the fluid sensor assembly; and wherein the processor is configured to process the signals received by the reader.

In some aspects, the one or more properties of the fluid can include at least one of flow rate, flow direction, and temperature.

The one or more output signals can be further indicative of a presence of air bubbles or occlusions in the system.

In some cases, the reader can be configured to generate an electromagnetic field configured to induce a current in a coil of the antenna, and wherein the current is configured to power at least one of the signal conditioning circuitry or the fluid sensor.

In some aspects, the reader and the antenna can be positioned about 25 cm or less from each other.

The fluid sensor can be configured to be removably positioned along a fluid flow path of an IV line extending from a fluid container to a patient.

In some cases, the fluid sensor can include a flow path and one or more sensing elements at least partially exposed to the flow path, and wherein the fluid is configured to flow through the flow path.

In some aspects, the signal conditioning circuitry can be configured to drive the one or more sensing elements and measure a change in sensing element resistance as the fluid flows through the flow path.

The fluid sensor assembly can include an analog-to-digital converter (ADC) configured to convert the signals processed by the signal conditioning circuitry to digital signals.

In some cases, the signal conditioning circuitry can include an analog front end (AFE).

In some aspects, the fluid sensor can include at least two substrates stacked on each other; a flow channel formed between the at least two substrates; a through substrate vias extending through at least a portion of at least one of the two substrates; one or more sensing elements along the flow channel; and a chip mounted to at least one of the two substrates; wherein the chip and the one or more sensing elements are in electrical communication via the through substrate vias.

### BRIEF DESCRIPTION OF THE DRAWINGS

FIGS. 1A-1C illustrate an example of a flow sensor glass sub-assembly.

FIGS. 2A and 2B illustrate an example of a flow sensor glass sub-assembly molded into a tube fitting.

FIG. 3 illustrates an example of a fitting for a flow sensor glass sub-assembly.

FIGS. 4A-4C illustrate another example of a flow sensor glass sub-assembly.

FIGS. 5A-5C illustrate an example of a flow sensor glass sub-assembly molded into a washer fitting.

FIG. 6 illustrates an example of a system including a fluid sensor for measuring fluid flow.

FIG. 7 illustrates an example of controller for a fluid sensor assembly.

### DETAILED DESCRIPTION

Flow sensors (also referred herein to as flow meters) can be used to measure and/or control the flow of a fluid, such as a gas and/or a liquid, through a tube and/or other conduit. In medical applications, flow sensors can be in fluid communication with a fluid source (gas tank, drug source, etc.) and a patient. For example, a flow sensor can be positioned between a patient and the fluid source via one or more tubes. As the fluid flows though the flow sensor, the flow sensor can measure a velocity and/or a volumetric flow rate of the liquid and/or gas as the fluid flows through the flow sensor. In some cases, the flow sensors described herein can be in communication with an external device, such as a cellphone, a tablet, a computer, and/or a central server, etc. The sensor data can be transmitted to the external device, which can beneficially allow health care providers to easily monitor and/or adjust the delivery of fluids. In some cases, the sensor data can be transmitted via a wireless network (e.g., near-field communication (NFC), Wi-Fi, Bluetooth, etc.).

The flow sensors described herein can be easily integrated into existing medical devices and/or medical procedures. For example, a flow sensor can include a glass sub-assembly. The dimensions of the glass sub-assembly can be easily adjusted to allow the flow sensor to be implemented in many applications. For example, a flow sensor can include a glass sub-assembly molded into a washer assembly for easy and convenient connection to one or more medical devices (e.g., via tubing).

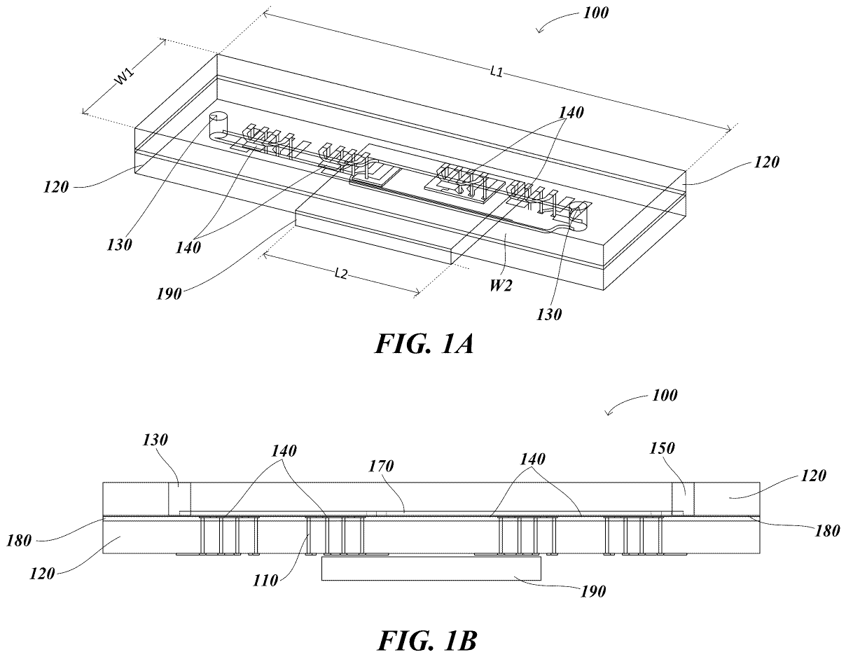

FIGS. 1A-1C show an example of a flow sensor glass sub-assembly. The glass sensor subassembly 100 can include one or more substrates 120, one or more resistors 140 (also referred to herein as sensing elements), one or more interconnects, and/or one or more insulating layers 180 (also referred to herein as passivation layers). In some cases, the one or more substrates 120 can include one or more glass layers. For example, the glass sensor sub-assembly 100 can include two glass layers. The one or more substrates 120 can be arranged in a stacked configuration (e.g., where a substrate is stacked on another substrate). For example, a first substrate can be stacked on top of a second substrate. In some cases, one or more through substrate vias can extend at least partially through the one or more glass layers. For example, one or more through glass vias (TGV) 110 can extend at least partially through the one or more glass layers. In cases where the one or more substrates 120 do not include glass layers, through substrate vias (TSV) can extend at least partially through the one or more substrates 120. The through glass vias (TGV) 110 and/or the through substrate vias (TSV) can act as an interconnect.

The one or more insulating layers 180 can include a polymer layer. For example, the polymer layer can include a polyimide (PI) layer. In some cases, the flow sensor glass sub-assembly 100 can include a thermally conductive PI layer over the one or more sensing elements on a bottom substrate (e.g., bottom glass layer). The flow sensor glass sub-assembly 100 can include a second thermally non-conductive PI layer under the one or more sensing elements on a top substrate (e.g., top glass layer). The one or more insulating layers 180, whether thermally conductive or non-conductive, can be positioned between the one or more substrates 120. The sensing elements 140 can be positioned between one or more insulating layers 180, whether thermally conductive or non-conductive. The one or more insulating layers 180 can beneficially prevent the one or more resistors 140 from shorting.

The one or more glass layers can form an inlet 130, an outlet 150, and a flow channel 170 extending between the inlet 130 and the outlet 150. In some cases, the inlet 130, the outlet 150, and the flow channel 170 may be formed by a single glass layer and/or by two or more glass layers. For example, a first glass layer can form at least one of the inlet 130, the outlet 150, and/or the flow channel 170, and a second glass layer can form the others. In some cases, the inlet 130 and the outlet 150 are formed on the same side of the glass sub-assembly 100. The inlet 130 and the outlet 150 can be formed on opposite halves of the glass sub-assembly 100. The inlet 130, the outlet 150, and/or the flow channel 170 can be formed into the one or more glass layers by etching the one or more glass layers. In some cases, the flow channel 170 can include a straight path. At least a portion of each of the one or more resistors 140 can be positioned along the flow channel 170. In some cases, the one or more resistors 140 can act as sensing elements to detect and/or measure one or more properties of a liquid and/or gas flowing through the flow channel 170.

The glass subassembly 100 can receive a liquid and/or a gas. For example, the liquid and/or gas can access the flow channel 170 via the inlet 130. As the liquid and/or gas flows through the flow channel 170, the glass sub-assembly 100 can measure one or more properties of the liquid and/or gas flowing through the flow channel 170. For example, the sensing elements (e.g., resistors) 140 along the flow channel 170 can measure a velocity, and/or a volumetric flow rate of the liquid and/or gas flowing through the flow channel 170.

A length L1 of the flow sensor glass sub-assembly can be from about 2 mm to about 10 mm. For example, the length L1 can be from about 3 mm to about 9 mm, from about 4 mm to about 8 mm, from about 5 mm to about 7 mm, and/or from 5.5 mm to about 6.5 mm. A width W1 of the flow sensor glass sub-assembly can be from about 0.5 mm to about 4 mm. For example, the width W1 can be from about 1 mm to about 3.5 mm, from about 1.2 mm to about 3 mm, from about 1.5 mm to about 2.5 mm, and/or from 1.8 mm to about 2.2 mm.

In some cases, the flow sensor glass sub-assembly can include a chip 190. In some cases, the chip 190 can include a transceiver and/or any other wireless communication controller for communication with a remote device, via cellular, Wi-fi, Bluetooth, etc. The chip 190 can include a near field communication (NFC) chip. For example, the glass sub-assembly 100 can include an NFC silicon flip chip attach. The NFC chip can be alternate connectorized In some cases, the chip 190 and the one or more resistors 140 can be in communication with each other via the one or more interconnects. A length L2 of the NFC chip can be from about 0.5 mm to about 4 mm. For example, the length L2 can be from about 1 mm to about 3.5 mm, from about 1.2 mm to about 3 mm, from about 1.5 mm to about 2.5 mm, and/or from 1.8 mm to about 2.2 mm. A width W2 of the NFC chip can be from about 0.5 mm to about 3.5 mm. For example, the width W2 can be from about 0.8 mm to about 3.2 mm, from about 1 mm to about 3 mm, from about 1.2 mm to about 2.5 mm, and/or from 1.5 mm to about 2 mm.

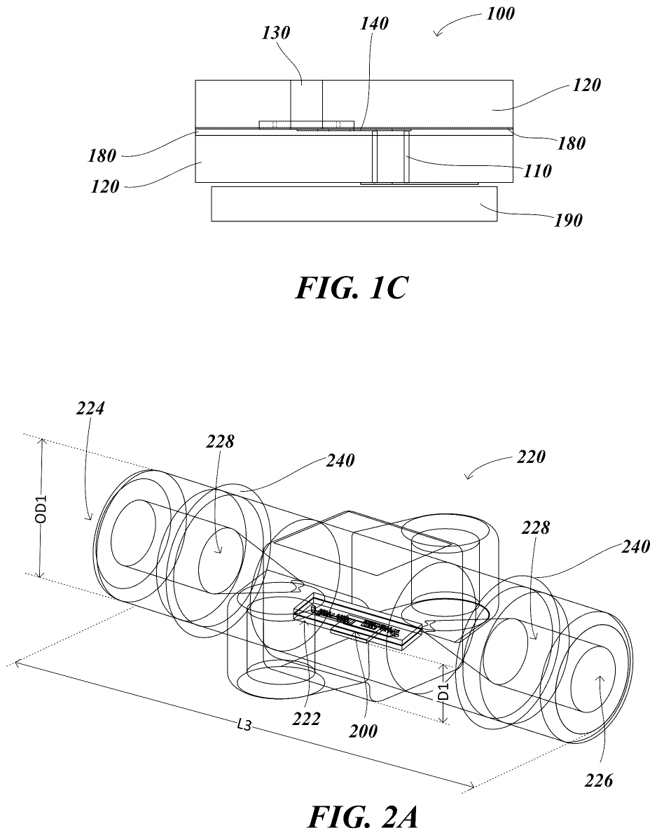

As shown in FIG. 2A, a flow sensor glass sub-assembly 200 can be positioned inside a tube fitting 220. The flow sensor glass sub-assembly 200 can be similar or identical to the flow sensor glass sub-assembly 200. In some cases, the glass sub-assembly 200 can be molded into the tube fitting 220. For example, the different components of the glass sub-assembly 200, including the one or more substrates, the one or more resistors, the one or more interconnects, one or more insulating layers (e.g., polyimide (PI) layers), and/or the through glass vias (TGV), can be fitted into the tube fitting 220 using injection molding. In some cases, the tube fitting 220 can include a fitting 222. The fitting 222 can be configured for convenient molding of the glass sub-assembly 200 to the tube fitting 220. Injection molding can allow for a highly customizable glass sub-assembly 200 and/or for a dimensionally stable glass-subassembly 200. This can beneficially allow the flow sensor glass sub-assembly 200 to provide precise readings while allowing for easy integration into devices.

The tube fitting 220 can include an inlet 224, an outlet 226, and a flow path 228 extending between the inlet 224 and the outlet 226. In some cases, the glass sub-assembly 200 can be positioned along the flow path 228 of the tube fitting 220. The inlet of the glass sub-assembly 200 can be positioned on the same side as the inlet 224 of the tube fitting 220. The outlet of the glass sub-assembly 200 can be positioned on the same side as the outlet 226 of the tube fitting 220.

As shown in FIG. 2B, the tube fitting 220 can include one or more barbs 240. The barbs 240 can facilitate attachment of the tube fitting 220 to tubing. The tubing can include tubing used in intravenous (IV), ventilator, drug delivery, etc., applications. One end of the tube fitting 220 can be connected to an inlet tubing and another end of the tube fitting 220 can be connected to an outlet tubing. The tube fitting 220 can include one or more indicators 250 to identify the flow within the tube fitting 220. For example, the one or more indicator 250 can include one or more visual indicators indicating the direction of the flow within the tube fitting 220 and/or the glass sub-assembly 200. The tube fitting 220 can receive a liquid and/or a gas from the inlet tubing. The liquid and/or gas can flow through the flow path 228 of the tube fitting 220. As the liquid and/or gas flows through the tube fitting 220, the glass sub-assembly 200 can measure the amount of liquid and/or gas flowing through the tube fitting 220. The liquid and/or gas may exit the glass sub-assembly 200 via the outlet of the glass-subassembly 200. The liquid and/or gas may exit the tube fitting 220 via the outlet 226 of the tube fitting 220 where it may continue to flow through the outlet tubing. In some cases, the flow channel of the glass sub-assembly 200 and the flow path 228 of the tube fitting 220 can be parallel to each other.

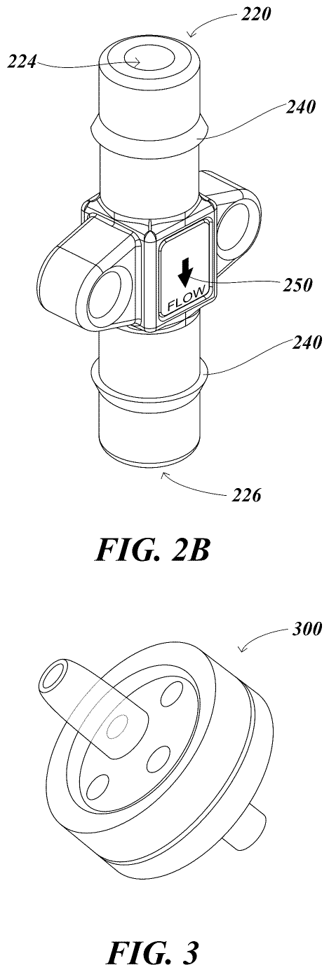

The tube fitting 220 can have a length L3 between about 10 mm and 50 mm. For example, the length L3 can be between about 15 mm to about 45 mm, between about 20 mm and 35 mm, between about 22 and about 32 mm, and/or between about 25 mm to about 30 mm. Each end of the tube fitting 220 can have an outer diameter OD1 from about 1 mm to about 10 mm. For example, the outer diameter OD1 can be between about 2 mm and 9 mm, between about 4 mm and about 8 mm, and/or between about 5 mm and 7 mm. In some cases, the tube fitting 220 can have dimensions similar or identical to those of commercially available intravenous (IV) check valves 300, an example of which is shown in FIG. 3.

In some cases, an NFC antenna of the NFC chip (e.g., the chip 190) can be oriented parallel to an antenna of an NFC reader. The NFC reader can be positioned on an exterior surface of the tube fitting 220. A distance D1 between the NFC chip and the NFC reader can be between about 0.5 mm and about 4 mm. For example, the distance D1 between the NFC chip and the NFC reader can be between about 0.8 mm and about 3.5 mm, between about 1 mm and about 3 mm, between about 1.5 mm and about 2.5 mm, and/or from about 1.8 mm to about 2.2 mm. The NFC chip can transmit data from the glass sub-assembly 200 to other devices. For example, the NFC chip can transmit flow measurements to an NFC reader compatible device. The NFC chip can also be used for power management of the flow sensor glass sub-assembly.

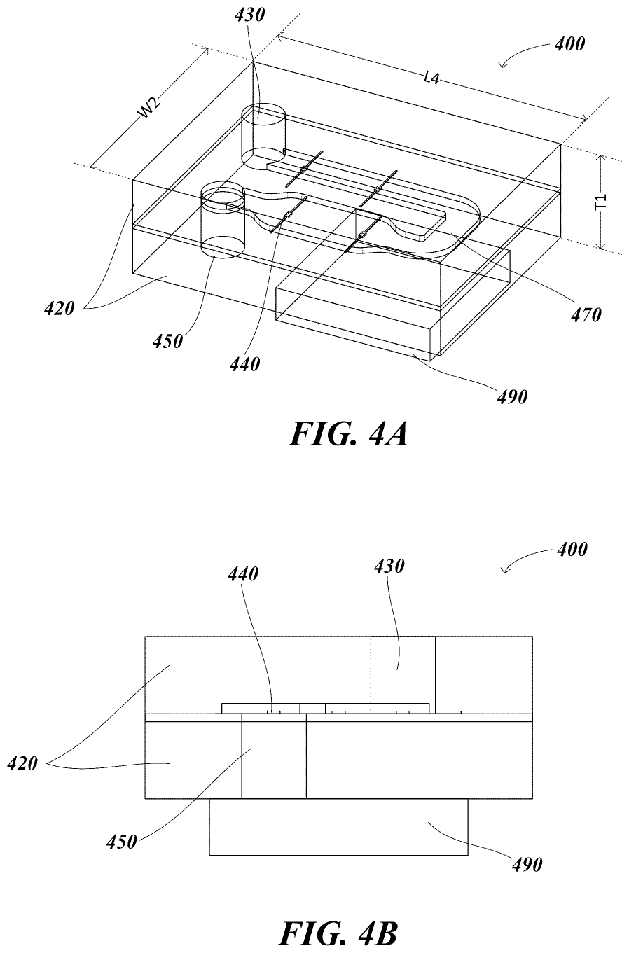

FIGS. 4A-4C show another example of a flow sensor glass sub-assembly. The glass sensor sub-assembly 400 can be similar or identical to the glass sensor sub-assembly 100 described in relation to FIGS. 1A-1C. For example, the glass sensor sub-assembly 400 shown in FIGS. 4A-4C can include one or more glass substrates 420 (e.g., glass layers), one or more resistors (which can include sensing elements, thermistors, resistance temperature detectors (RTD), thermocouples, and/or analog or digital thermometer integrated circuits (IC)) 440, one or more interconnects, one or more insulating layers (e.g., polyimide (PI) layers) 480, a through glass vias (TGV) 410, and/or a chip 490. Unlike the glass sensor sub-assembly of FIGS. 1A-1C, the glass sensor sub-assembly 400 of FIGS. 4A-4C can include an inlet 430 and an outlet 450 formed on opposite sides of the glass sensor sub-assembly 400. Further, the inlet 430 and the outlet 450 can be formed on the same half of the glass sensor sub-assembly 400. The inlet 430 can be formed on a first layer of glass, and the outlet 450 can be formed on a second layer of glass. In some cases, one or more through glass vias (TGV) 410 can extend between the first layer of glass and the second layer of glass. The first and second glass layers can form a flow channel 470. The flow channel 470 can extend between the inlet 430 and the outlet 450. In some cases, the flow channel 470 can include a curved path, as shown in FIG. 4C. One or more sensing structures 440 can be positioned between the glass layers and/or along the flow channel 470.

A length L4 of the glass sensor sub-assembly 400 can be from about 0.5 mm to about 4 mm. For example, the length LA can be from about 1 mm to about 3.5 mm, from about 1.2 mm to about 3 mm, from about 1.5 mm to about 2.5 mm, and/or from 1.8 mm to about 2.2 mm. A width W2 of the glass sensor sub-assembly 400 can be from about 0.5 mm to about 3 mm. For example, the width W2 can be from about 0.8 mm to about 2.8 mm, from about 1 mm to about 2.5 mm, from about 1.2 mm to about 2 mm, and/or from about 1.4 mm to about 1.6 mm. A thickness T1 of the glass sensor sub-assembly can be from about 0.1 mm to about 1.1 mm. For example, the thickness T1 can be from about 0.2 mm to about 1 mm, from about 0.3 mm to about 0.9 mm, from about 0.4 mm to about 0.8 mm, and/or from about 0.5 mm to about 0.7 mm.

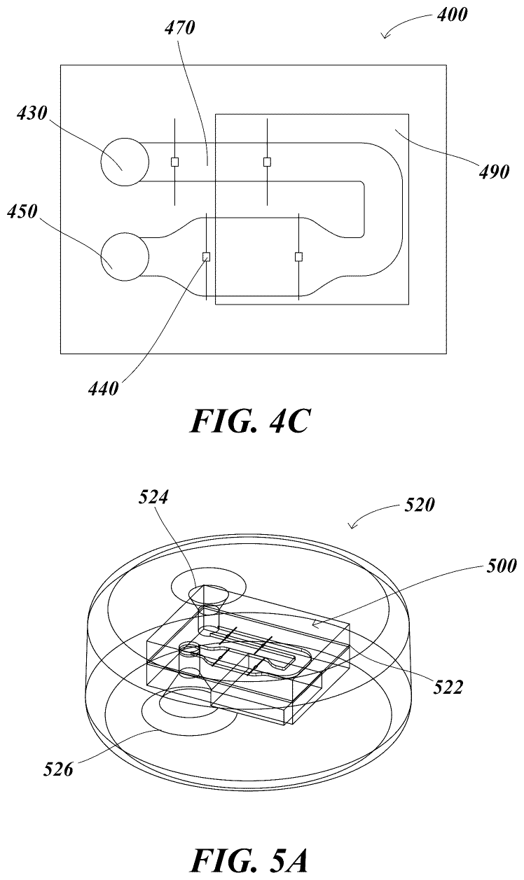

As shown in FIGS. 5A-5C, a flow sensor glass sub-assembly 500 can be positioned inside a washer fitting 520. The flow sensor glass sub-assembly 500 can be similar or identical to the flow sensor glass sub-assemblies 100, 200, and/or 400. In some cases, the glass sub-assembly 500 can be molded into the washer fitting 520. For example, the different components of the glass sub-assembly 500, including the one or more glass layers, the one or more resistors, the one or more interconnects, one or more insulating layers (e.g., polyimide (PI) layers), and/or the through glass vias (TGV), can be fitted into the washer fitting 520 using injection molding. In some cases, the washer fitting 520 can include a fitting 522. The fitting 522 can be configured for convenient molding of the glass sub-assembly 500 to the washer fitting 520.

The washer fitting 520 can include an inlet 524, an outlet 526, and a flow channel 528 extending between the inlet 524 and the outlet 526. In some cases, the glass sub-assembly 500 can be positioned along the flow channel 528 of the washer fitting 520. The inlet of the glass sub-assembly 500 can be positioned on the same side as the inlet 524 of the washer fitting 520. The outlet of the glass sub-assembly 500 can be positioned on the same side as the outlet 526 of the washer fitting 520.

The washer fitting 520 can have an outer diameter OD2 from about 1 mm to about 8 mm. For example, the outer diameter OD2 can be from about 2 mm to about 7 mm, from about 2.5 mm to about 6 mm, from about 3 mm to about 5 mm, and/or from about 3.5 mm to about 4.5 mm. The washer fitting can have an thickness T2 from about 0.5 mm to about 2.5 mm. For example, the thickness T2 can be from about 0.8 mm to about 2.2 mm, from about 1 mm to about 2 mm, from about 1.2 mm to about 1.8 mm, and/or from about 1.4 mm to about 1.6 mm.

Liquid and/or gas can flow through the flow channel 528 of the washer fitting 520. As the liquid and/or gas flows through the washer fitting 520, the glass sub-assembly 500 can measure the amount of liquid and/or gas flowing through the washer fitting 520. For example, the sensing elements (e.g., resistors) along the flow channel 528 can measure a velocity, and/or a volumetric flow rate of the liquid and/or gas flowing through the flow channel 528. The liquid and/or gas may exit the glass sub-assembly 500 via the outlet of the glass-subassembly 500. The liquid and/or gas may exit the washer fitting 520 via the outlet 526 of the tube fitting 520.

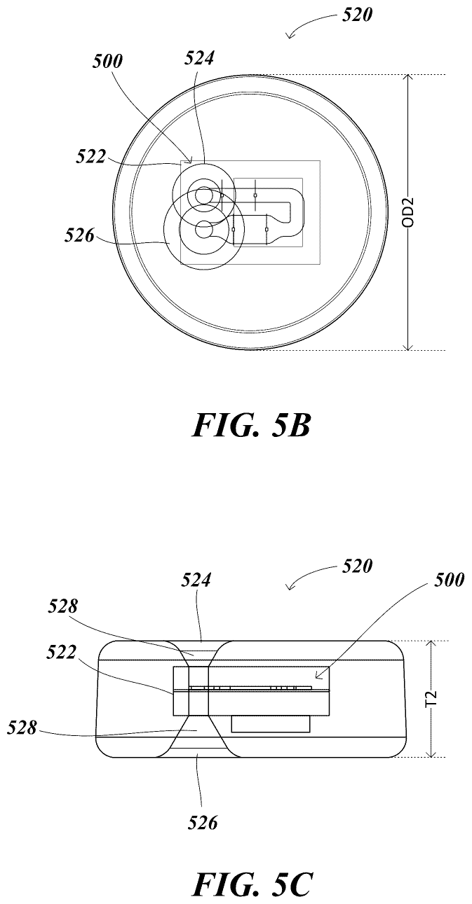

FIG. 6 shows an example of a system including a wireless fluid sensor for inline liquid sensing. The system can be implemented in medical devices including, but not limited to, intravenous (IV) infusion pumps, dialysis machines, catheters, etc. As shown in FIG. 6, the system 600 can include a fluid sensor assembly 620 having a fluid sensor 630 and a first processor 640, and/or a second processor 660. The fluid sensor assembly 620 can be similar or identical to the flow sensor glass sub-assemblies 100, 200, 400, and/or 500. The fluid sensor 630 can include sensing elements such as thermistors, resistance temperature detectors (RTD), thermocouples, and/or analog or digital thermometer integrated circuits (IC). In some cases, the first processor 640 and the second processor 660 can be in communication with each other. For example, as further described below, the first processor 640 and the second processor 660 can be in communication with each other via a wireless network (e.g., near-field communication (NFC), Wi-Fi, Bluetooth, etc.). Each of the first processor 640 and/or the second processor 660 can include an antenna, a reader, a transmitter, or a combination thereof to communicated with each other. In some cases, the second processor 660 can be in communication with and/or control operation of an infusion pump 690.

The fluid sensor assembly 620 can be in fluid communication with a container 670 via a first fluid line 682. The container 670 can store fluids such as drugs, saline, medications, etc. The first fluid line 682 can facilitate flow of fluids from the container 670 to the fluid sensor assembly 620. The fluid sensor assembly 620 can be in fluid communication with a patient via a second fluid line 684. The second fluid line 684 can facilitate delivery of the fluids from the container 670 to a patient. As way of example, the fluid from the container 670 can flow through the first fluid line 682, a flow channel extending through the fluid sensor 630 (e.g., flow channel 470), and the second fluid line 684, for infusion into a patient.

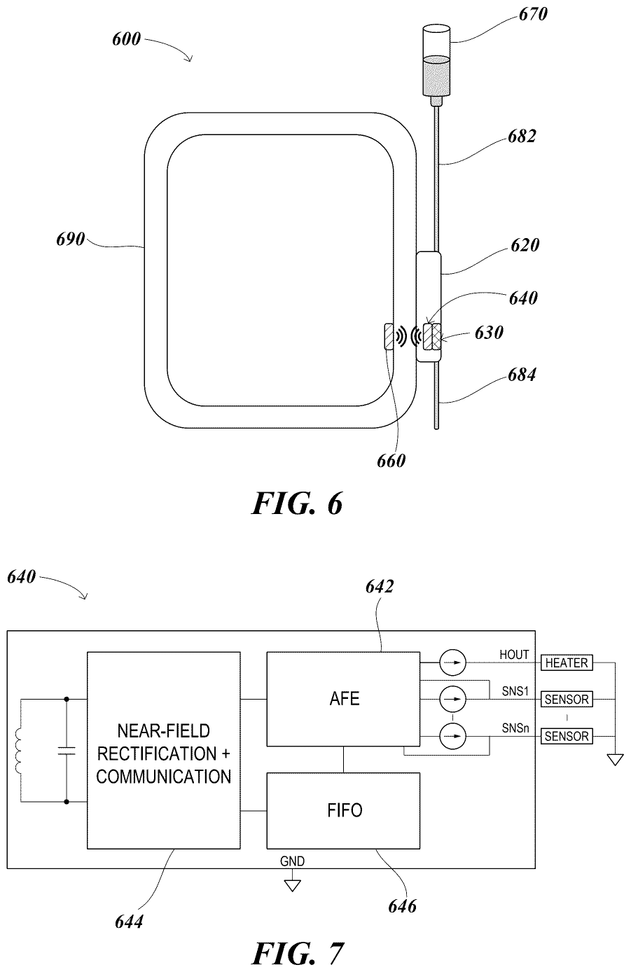

As shown in FIG. 7, the first processor 640 can include a signal conditioning circuitry. For example, the first processor can include an analog front end (AFE) 642. The first processor can also include a near field communication (NFC) chip 644, and/or a data buffer 646. The AFE 642 can receive sensing data (e.g., analog signals) from the fluid sensor 630. The AFE 642 can filter, amplify, and/or condition the sensing data received from the fluid sensor 630. The data processed by the AFE 642 can be converted from analog to digital through an analog-to-digital converter (ADC). The ADC can be integrated into the first processor 640.

The first processor 640 can control operational settings of the fluid sensor 630. For example, the AFE 642 of the first processor 640 can drive one or more thermistors (e.g., resistors 440) of the fluid sensor 630. In some cases, the one or more thermistors can include one or more thermistor pairs having a heater thermistor and a corresponding monitor thermistor. The AFE 642 can drive the one or more thermistors of the fluid sensor 630 by supplying a current and/or voltage. The current and/or voltage supplied by the AFE 642 can heat each of the one or more thermistors. In some cases, the current and/or voltage can be supplied to the one or more heater thermistors. As fluid flows through the fluid sensor 630, the fluid can dissipate at least some of the heat from the one or more heater thermistors. The one or more corresponding monitor thermistors can be positioned downstream of the heater thermistors. As the fluid flows though the monitor thermistors, a resistance of the one or more monitor thermistors can change based on the temperature of the fluid. The change in resistance of the thermistors can be processed by the first processor 640 and/or the second processor 660 to calculate the flow rate, direction, and/or temperature of the fluid flowing through the fluid sensor 630. As further described herein, the change in resistance of the thermistors can be processed by the first processor 640 and/or the second processor 660 to detect air bubbles and/or occlusions in the fluid lines.

In some cases, the data buffer 646 can manage the data received from the fluid sensor 630. The data buffer 646 can include a first-in, first-out (FIFO) buffer that can store the data received from the fluid sensor 630. The FIFO can store the data received from the fluid sensor 630 in the order in which the data is received. The data buffer 646 can allow the AFE 642 to read and process the data received from the fluid sensor 630 at the same or different pace than a data processing rate of the fluid sensor 630. The data buffer 646 can beneficially prevent or reduce data loss.

In some cases, the NFC chip 644 of the first processor 640 can facilitate communication between the fluid sensor 630 and the first processor 640 and/or between the second processor 660 and the first processor 640. For example, the data conditioned by the AFE 642 can be sent to the second processor 660. The NFC chip 644 can operate over short-range, low-power radio waves, which can beneficially allow data to be transmitted with high reliability. The data sent by the NFC chip 644 can be received by an NFC antenna of the second processor 660. Data exchange between the first processor 640 and the second processor 660 can occur, for example, when the NFC antenna of the first processor 640 and the NFC antenna of the second processor 660 are brought into proximity (e.g., when the NFC antennae are positioned between about 0.1 cm and about 12 cm from each other). The data received by the second processor 660 can be decoded and/or processed by an NFC reader and/or an edge processor of the second processor 660.

Power for the AFE 642 and/or the one or more thermistors of the fluid sensor 630 can be supplied by the second processor 660. For example, the NFC reader of the second processor 660 can wirelessly transfer energy to the NFC antenna of the first processor 640. This can beneficially allow the fluid sensor assembly to operate without a battery. The NFC reader of the second processor 660 can generate an electromagnetic field when the NFC antenna of the first processor 640 is in range (e.g., when the first processor 640 is about 25 cm or less from the second processor 660). The electromagnetic field generated by the NFC reader can. The harvested current can be used to power the AFE 642 and/or the one or more thermistors.

The NFC reader of the second processor 660 can forward the data received from the NFC chip 644 to the edge processor of the second processor 660. The edge processor of the second processor 660 can process the data received from the NFC reader to calculate the flow rate, direction, and/or temperature of the fluid flowing through the fluid sensor 630 and/or to detect air bubbles and/or occlusions in the fluid lines connected to the fluid sensor assembly 620. The edge processor of the second processor 660 can, in some cases, control operation of the infusion pump 690. For example, based on the detected flow rate, direction, and/or temperature of the fluid flowing through the fluid sensor assembly 620, the second processor 660 can adjust the operational settings of the infusion pump 690. As a non-limiting example, if the detected flow rate of the fluid is below a predefined fluid administration rate, the second controller can adjust the operational settings of the infusion pump 690 to increase the fluid flow rate. As another example, if the detected flow rate of the fluid is above a predefined fluid administration rate, the second controller can adjust the operational settings of the infusion pump 690 to decrease the fluid flow rate. In some cases, the second controller can stop operation of the infusion pump 690 when, for example, air bubbles and/or occlusions are detected.

The fluid sensor assembly 620 and/or the second processor 660 can include a transceiver and/or a wireless communication controller for communication with a remote device (e.g., cellphone, tablet, computer, etc.), via cellular, Wi-fi, Bluetooth, etc. The transceiver and/or wireless communication controller can facilitate data exchange between the remote device and the fluid sensor assembly 620 and/or the second processor 660. In some cases, the sensing data detected by the fluid sensor assembly 620 can be displayed on a user interface of the infusion pump 690 and/or the remote device. The data displayed on the infusion pump 690 and/or the remote device can be updated in real time.

Unless the context clearly requires otherwise, throughout the description and the claims, the words “comprise,” “comprising,” “include,” “including,” and the like are to be construed in an inclusive sense, as opposed to an exclusive or exhaustive sense; that is to say, in the sense of “including, but not limited to.” The word “coupled,” as generally used herein, refers to two or more elements that may be either directly coupled to each other, or coupled by way of one or more intermediate elements. Likewise, the word “connected,” as generally used herein, refers to two or more elements that may be either directly connected, or connected by way of one or more intermediate elements. Additionally, the words “herein,” “above,” and words of similar import, when used in this application, shall refer to this application as a whole and not to any particular portions of this application. Where the context permits, words in the above Detailed Description using the singular or plural number may also include the plural or singular number respectively. Where the context permits, the word “or” in reference to a list of two or more items is intended to cover all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments.

While certain embodiments have been described, these embodiments have been presented by way of example, and are not intended to limit the scope of the disclosure. Indeed, the novel methods, apparatus, and systems described herein may be embodied in a variety of other forms. Furthermore, various omissions, substitutions and changes in the form of the methods, apparatus, and systems described herein may be made without departing from the spirit of the disclosure. For example, device components described herein may be deleted, moved, added, subdivided, combined, and/or modified. Each of these device components may be implemented in a variety of different ways. The accompanying claims and their equivalents are intended to cover any such forms or modifications as would fall within the scope and spirit of the disclosure.

The above description is intended to be illustrative and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

1. A system for measuring one or more properties of a fluid, the system comprising:
a fluid sensor assembly comprising a fluid sensor and an antenna, wherein the fluid sensor is configured to be removably positioned along a fluid flow line;
a processor in wireless communication with the fluid sensor assembly and comprising a reader;
wherein the fluid sensor is configured to output one or more output signals indicative of one or more properties of the fluid flowing through the fluid sensor;
wherein the antenna of the fluid sensor assembly is configured to transmit the output signals to the reader of the processor; and
wherein the processor is configured to process the signals received by the reader.

2. The system of claim 1, wherein the fluid sensor comprises a flow path in communication with the fluid flow line, and one or more resistor pairs at least partially exposed to the flow path.

3. The system of claim 2, wherein the fluid sensor assembly is further configured to drive the one or more resistor pairs and measure a change in resistance as the fluid flows through the flow path.

4. The system of claim 1, wherein the one or more properties of the fluid comprise at least one of flow rate, flow direction, and temperature.

5. The system of claim 1, wherein the one or more output signals are further indicative of a presence of air bubbles or occlusions in the system.

6. The system of claim 1, wherein the reader is configured to generate an electromagnetic field configured to induce a current in a coil of the antenna, and wherein the current is configured to power the fluid sensor.

7. The system of claim 1, wherein the reader and the antenna are positioned about 25 cm or less from each other.

8. The system of claim 1, wherein the fluid sensor assembly further comprises an analog-to-digital converter (ADC) configured to convert the output signals to digital signals.

9. The system of claim 1, further comprising an infusion pump, and wherein the processor is positioned inside the infusion pump and is further configured to control operational settings of the infusion pump based at least in part on the processed signals.

10. (canceled)

11. The system of claim 1, wherein the fluid sensor comprises
at least two substrates stacked on each other;
a flow channel formed between the at least two substrates;
a through substrate vias extending through at least a portion of at least one of the two substrates;
one or more sensing elements along the flow channel; and
a chip mounted to at least one of the two substrates;
wherein the chip and the one or more sensing elements are in electrical communication via the through substrate vias.

12. A system for measuring one or more properties of a fluid, the system comprising:
an infusion pump;
a fluid container configured to contain a fluid;
a fluid sensor assembly comprising,
a fluid sensor comprising a flow path and one or more sensing elements at least partially exposed to the flow path, wherein the fluid from the fluid container is configured to flow through the flow path, and
a first processor in communication with the fluid sensor and comprising a signal conditioning circuitry, and an antenna;
a first fluid line configured to place the fluid container in communication with the flow path of the fluid sensor;
a second fluid line configured to place flow path of the fluid sensor in communication with a patient; and
a second processor in communication with the fluid sensor assembly and comprising a reader, wherein the second processor is positioned on the infusion pump;
wherein the fluid sensor is configured to output one or more output signals representative of a change in resistance of the one or more sensing elements, and wherein the change of resistance of the one or more sensing element is indicative of one or more properties of the fluid flowing through the flow path of the fluid sensor;
wherein the signal conditioning circuitry is configured to receive and process the one or more output signals from the fluid sensor,
wherein the antenna of the first processor is configured to transmit the signals processed by the signal conditioning circuitry across the infusion pump;
wherein the reader of the second processor is configured to receive the signals transmitted by the antenna of the first processor; and
wherein the second processor is configured to process the signals received by the reader.

13. The system of claim 12, wherein the one or more sensing elements comprise one or more thermistor pairs.

14. The system of claim 12, wherein the signal conditioning circuitry comprises an analog front end (AFE).

15. The system of claim 12, wherein the antenna of the first processor comprises a near-field communication (NFC) antenna.

16. The system of claim 12, wherein the reader of the second processor comprises a near-field communication (NFC) reader.

17. (canceled)

18. (canceled)

19. (canceled)

20. (canceled)

21. (canceled)

22. The system of claim 12, wherein the fluid sensor is configured to be removably positioned along a fluid flow path of an IV line extending from a fluid container to a patient.

23. (canceled)

24. (canceled)

25. The system of claim 12, wherein the fluid sensor comprises
at least two substrates stacked on each other, wherein the flow path is formed between the at least two substrates;
a through substrate vias extending through at least a portion of at least one of the two substrates; and
a chip mounted to at least one of the two substrates;
wherein the chip and the one or more sensing elements are in electrical communication via the through substrate vias.

26. A system for measuring one or more properties of a fluid, the system comprising:
an infusion pump;
a fluid sensor assembly comprising a fluid sensor, a fluid sensor signal conditioning circuitry, and an antenna;
a processor in wireless communication with the fluid sensor assembly and comprising a reader, wherein the processor is positioned inside the infusion pump;
wherein the fluid sensor is configured to output one or more output signals indicative of one or more properties of a fluid flowing through the fluid sensor;
wherein the signal conditioning circuitry is configured to receive and condition the one or more output signals from the fluid sensor, wherein the antenna of the fluid sensor assembly is configured to transmit the signals processed by the signal conditioning circuitry across a wall of the infusion pump;
wherein the reader of the processor is configured to receive the signals transmitted by the antenna of the fluid sensor assembly; and
wherein the processor is configured to process the signals received by the reader.

27. (canceled)

28. (canceled)

29. (canceled)

30. (canceled)

31. (canceled)

32. The system of claim 26, wherein the fluid sensor comprises a flow path and one or more sensing elements at least partially exposed to the flow path, and wherein the fluid is configured to flow through the flow path.

33. (canceled)

34. (canceled)

35. (canceled)

36. The system of claim 26, wherein the fluid sensor comprises
at least two substrates stacked on each other;
a flow channel formed between the at least two substrates;
a through substrate vias extending through at least a portion of at least one of the two substrates;
one or more sensing elements along the flow channel; and
a chip mounted to at least one of the two substrates;
wherein the chip and the one or more sensing elements are in electrical communication via the through substrate vias.
